(19) United States
(12) Patent Application Publication
(10) Pub. No.: US 2026/0020850 A1
(43) Pub. Date: Jan. 22, 2026

(54) SUTURE LOCKING SYSTEMS AND METHODS THAT UTILIZE LOCKING FERRULES WITH ONE-WAY LOCKING MECHANISMS

(71) Applicant: ARTHREX, INC., Naples, FL (US)

(72) Inventor: Thomas Dooney, Naples, FL (US)

(21) Appl. No.: 19/272,309

(22) Filed: Jul. 17, 2025

Related U.S. Application Data
(60) Provisional application No. 63/674,025, filed on Jul. 22, 2024.

(51) Int. Cl. A61B 17/04

(52) U.S. Cl. CPC A61B 17/0401; A61B 2017/0462

(57) ABSTRACT

Systems and methods are provided for performing tensionable knotless surgical procedures. Suture locking systems that provide one or more one-way locking mechanisms may be utilized for tensioning and locking one or more strands of suture during the surgical procedures. The one-way locking mechanisms may be established by one or more locking barbs provided within the suture locking systems.

### CROSS-REFERENCE TO RELATED APPLICATIONS

This application claims the benefit of U.S. Provisional Application No. 63/674,025, which was filed on Jul. 22, 2024 and is incorporated herein by reference in its entirety.

### BACKGROUND

This disclosure relates to the field of surgery, and more particularly to various suture locking systems that are capable of locking suture when performing a variety of tensionable knotless surgical repairs.

Repetitive trauma to a joint, such as a knee, ankle, hip, elbow, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless surgical procedures, such as tissue repairs, for example. A one-way locking mechanism may be utilized when performing the tensionable knotless surgical procedures for tensioning and locking one or more strands of suture.

An exemplary suture locking system may include, inter alia, a surgical fixation device including an opening, a locking ferrule insertable within the opening and including a one-way locking mechanism provided within a cannulation of the locking ferrule, and a suture received through the cannulation. The one-way locking mechanism is configured to allow the suture to be tensioned in a first direction while preventing the suture from moving in a second direction.

Another exemplary suture locking system may include, inter alia, a suture anchor including a body portion and a suture portion connected to the body portion, a locking ferrule received within a lumen of a tail of the suture portion and including a one-way locking mechanism provided within a cannulation of the locking ferrule, and a suture received through the cannulation. The one-way locking mechanism is configured to allow the suture to be tensioned in a first direction and prevent the suture from moving in a second direction.

Yet another exemplary suture locking system may include, inter alia, a suture anchor including a sheath having an internal bore, a locking ferrule including a one-way locking mechanism, and a suture received through the internal bore of the sheath and the locking ferrule.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary suture locking system.

FIG. 2 is an exploded view of the suture locking system of FIG. 1.

FIG. 3 is a cross-sectional view through section 3-3 of FIG. 2.

FIG. 4 illustrates a plurality of rows of locking barbs of a locking ferrule of the suture locking system of FIGS. 1-3.

FIG. 5 illustrates an exemplary suture of the suture locking system of FIGS. 1-3.

FIG. 6 schematically illustrates a surgical method for performing a tissue repair.

FIGS. 7 and 8 illustrate another exemplary suture locking system.

FIG. 9 illustrates yet another exemplary suture locking system.

### DETAILED DESCRIPTION

This disclosure relates to systems and methods for performing tensionable knotless surgical procedures. A suture locking system may include one or more one-way locking mechanisms for tensioning and locking one or more strands of suture during a surgical procedure. These and other features of this disclosure are described in further detail below.

An exemplary suture locking system may include, inter alia, a surgical fixation device including an opening, a locking ferrule insertable within the opening and including a one-way locking mechanism provided within a cannulation of the locking ferrule, and a suture received through the cannulation. The one-way locking mechanism is configured to allow the suture to be tensioned in a first direction while preventing the suture from moving in a second direction.

In any further embodiment, the surgical fixation device is a surgical button.

In any further embodiment, the surgical fixation device is a bone plate.

In any further embodiment, the suture is connected to a second surgical fixation device.

In any further embodiment, the second surgical fixation device is a suture anchor.

In any further embodiment, the locking ferrule includes a cap portion and a barrel portion. The barrel portion is received through the opening, and the cap portion is oversized relative to the opening.

In any further embodiment, the one-way locking mechanism is established by a plurality of locking barbs provided within the cannulation.

Another exemplary suture locking system may include, inter alia, a suture anchor including a body portion and a suture portion connected to the body portion, a locking ferrule received within a lumen of a tail of the suture portion and including a one-way locking mechanism provided within a cannulation of the locking ferrule, and a suture received through the cannulation. The one-way locking mechanism is configured to allow the suture to be tensioned in a first direction and prevent the suture from moving in a second direction.

In any further embodiment, a plurality of locking barbs protrude inwardly from an inner diameter wall of the locking ferrule, and a plurality of barbs are provided that protrude outwardly from an outer diameter wall of the locking ferrule to engage an interior wall of the tail.

In any further embodiment, the suture extends through a cannulated passage of the body portion of the suture anchor.

In any further embodiment, a knot of the suture portion secures the suture portion to the body portion of the suture anchor.

In any further embodiment, the knot and the tail are located at opposite ends of the body portion.

Yet another exemplary suture locking system may include, inter alia, a suture anchor including a sheath having an internal bore, a locking ferrule including a one-way locking mechanism, and a suture received through the internal bore of the sheath and the locking ferrule.

In any further embodiment, the one-way locking mechanism allows the suture to be tensioned in a first direction and prevents the suture from moving in a second direction relative to the locking ferrule.

In any further embodiment, the one-way locking mechanism is established by a plurality of locking barbs provided within a cannulation of the locking ferrule.

In any further embodiment, the sheath is comprised exclusively of soft, suture-based materials.

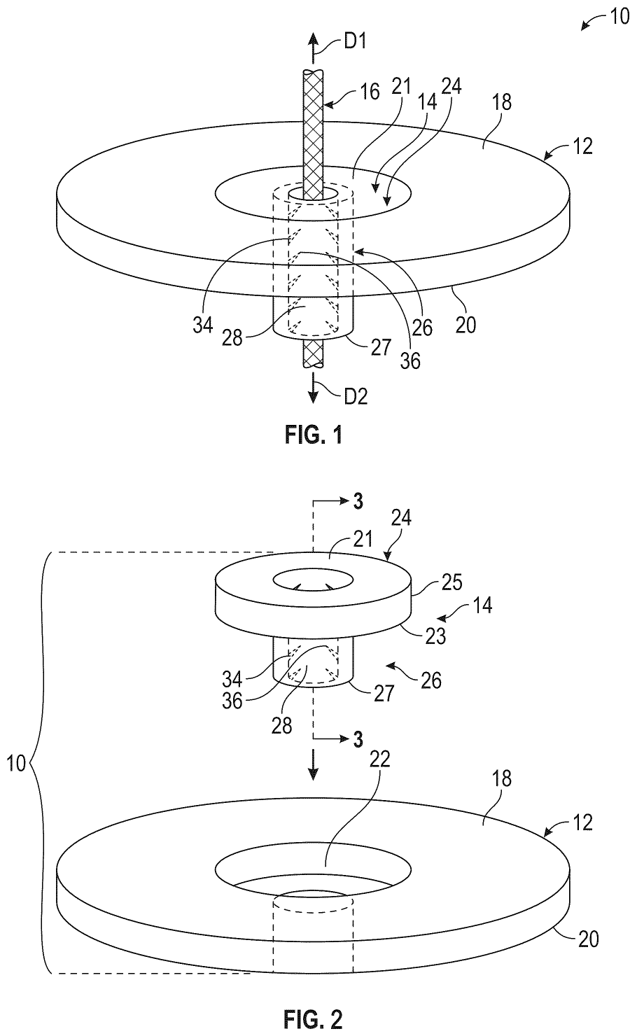

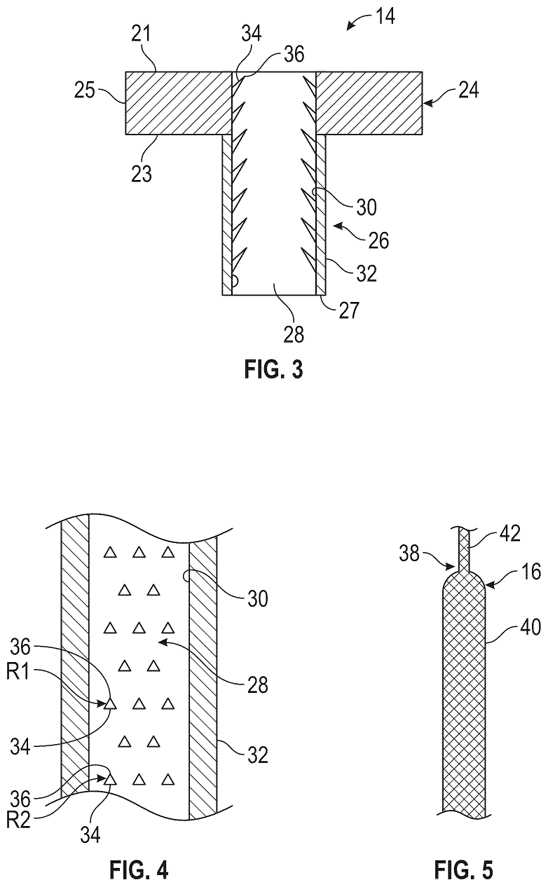

FIGS. 1-4 illustrate various aspects of an exemplary suture locking system 10 that can be used when performing various tensionable knotless surgical procedures. For example, the suture locking system 10 could be utilized during surgical methods for compressing tissue to another tissue (e.g., soft tissue to bone, soft tissue to another soft tissue, etc.). The suture locking system 10 could be used during a variety of orthopedic surgical repairs, including but not limited to cerclage procedures, AC joint repairs, syndesmosis repairs, and fracture repairs, among many others.

The suture locking system 10 may include a surgical fixation device 12, a locking ferrule 14, and at least one suture 16. The surgical fixation device 12 may be a surgical button or a surgical bone plate, for example. The surgical fixation device 12 may be made from any biocompatible material or combination of materials, including but not limited to polymeric materials, metallic materials, or embroidered materials.

The surgical fixation device 12 may include a top surface 18 and a bottom surface 20. One or more openings 22 may be formed through the surgical fixation device 12 such that each opening 22 penetrates through both the top surface 18 and the bottom surface 20. The opening 22 may be sized to receive the locking ferrule 14.

The locking ferrule 14 may include a cap portion 24 and a tube or barrel portion 26 that protrudes from the cap portion 24. The cap portion 24 and the barrel portion 26 may be formed together as part of a unitary structure of the locking ferrule 14. In an embodiment, the cap portion 24 and the barrel portion 26 are arranged to provide a top hat-like appearance of the locking ferrule 14. The specific size, shape, and material make-up of the locking ferrule 14 are not intended to limit this disclosure.

The cap portion 24 of the locking ferrule 14 may include a top surface 21, a bottom surface 23, and a side wall 25 that extends between the top surface 21 and the bottom surface 23. The barrel portion 26 may protrude outwardly from the bottom surface 23 of the cap portion 24.

The locking ferrule 14 may be positioned within the opening 22 of the surgical fixation device 12 such that the cap portion 24 is received in abutting contact with the top surface 18 of the surgical fixation device 12 and the barrel portion 26 extends at least partially through the opening 22. The top surface 21 of the cap portion 24 may or may not be positioned flush with the top surface 18 of the surgical fixation device 12 once the locking ferrule 14 is fully seated within the opening 22. A distal tip 27 of the barrel portion 26 may extend to a position that is flush with the bottom surface 20 of the surgical fixation device 12 or could protrude outwardly thereof once the locking ferrule 14 is fully seated within the opening 22.

The cap portion 24 is oversized relative to the opening 22. The cap portion 24 can thus act as a washer or buttress to prevent the locking ferrule 14 from being pulled through the surgical fixation device 12 when the suture locking system 10 is used during a surgical method.

A cannulation 28 may extend through the cap portion 24 and the barrel portion 26 of the locking ferrule 14. The cannulation 28 may each establish an internal passageway for accommodating one or more strands of the suture 16. The cannulation 28 may be circumscribed by an interior wall 30 of the locking ferrule 14. The interior wall 30 may be established by both the cap portion 24 and the barrel portion 26. An outer diameter wall 32 of the locking ferrule 14 could provide a smooth surface or could alternatively include threads, barbs, or other mating features for facilitating fixation within the opening 22 of the surgical fixation device 12.

A plurality of locking barbs 34 may protrude inwardly from the interior wall 30. The locking barbs 34 may therefore occupy at least a portion of the open space of the cannulation 28. In an embodiment, the locking barbs 34 are integrally formed (e.g., molded) features of the body of the locking ferrule 14. The locking barbs 34 may be provided along an entire length of the cannulation 28 or at only select portions thereof. The locking barbs 34 may be either rigid or flexible structures.

The locking barbs 34 may be arranged in multiple rows along the length of the cannulation 28. For example, the locking barbs 34 may be arranged in a least a first row R1 and a second row R2 (see FIG. 4). In an embodiment, the locking barbs 34 of the second row R2 are staggered relative to the locking barbs 34 of the first row R1 (see FIG. 4).

Each locking barb 34 may include a sharp or pointed tip 36, and, in an exemplary implementation, each locking barb 34 may be angled in a direction toward the top surface 21 of the cap portion 24 of the locking ferrule 14. The locking barbs 34 are therefore configured to establish a one-way locking mechanism that permits one or more sutures 16 to pass through the cannulation 28 in a first direction D1 while preventing the suture(s) 16 from being tensioned or otherwise moved in a second direction D2. The staggered relationship of the rows of locking barbs 34 can maximize engagement with the suture 16 once it is passed through the cannulation 28.

The suture 16 may be FiberWire®, FiberTape®, or any other suitable suture product. FiberWire® and FiberTape® are suture products marketed and sold by Arthrex, Inc. However, other suture products could be utilized for the suture 16 within the scope of this disclosure. The size and type of suture utilized in conjunction with the suture locking system 10 are not intended to limit this disclosure.

The suture 16 may include a varying thickness. For example, the suture 16 may include one or more tapered regions 38 where the suture 16 transitions between a thickened section 40 and a thinned section 42 (see FIG. 5). The thinned sections 42 can facilitate passing the suture 16 through the cannulations 28, such as via a suitable suture loader that can be provided as part of the suture locking system 10, and the thickened sections 40 can provide greater surface area for the locking barbs 34 to engage in order to sufficiently lock the suture 16 after tensioning the suture 16 relative to the locking ferrule 14.

In an embodiment, the thickened sections 40 of the suture 16 are about twice as thick as the thinned sections 42. However, other ratios (e.g., 1.5:1, 3:1, etc.) between the relative thicknesses (e.g., outer diameters) of the thickened sections 40 and the thinned sections 42 are contemplated within the scope of this disclosure.

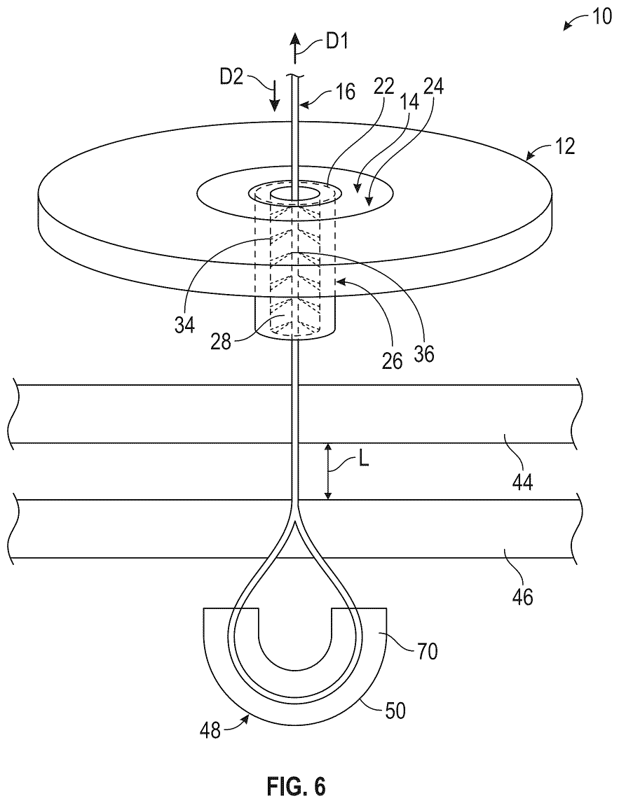

The suture locking system 10 described above and shown in FIGS. 1-5 may be utilized to tension and knotlessly fixate the one or more sutures 16 as part of various surgical methods. FIG. 6 schematically illustrates one such surgical method that can be performed for compressing or fixating a first tissue 44 (e.g., a soft tissue or bone) relative to a second tissue 46 (e.g., another soft tissue or bone).

An additional surgical fixation device 48, here a suture anchor, may first be implanted within or otherwise positioned relative to the second tissue 46 during the surgical method. The surgical fixation device 48 may be considered an additional subcomponent of the suture locking system 10 in this implementation. The suture 16 may be attached to a body 50 of the surgical fixation device 48 prior to or subsequent to implantation of the surgical fixation device 48 relative to the second tissue 46. In an embodiment, the body 50 is a sheath made exclusively of soft, suture-based materials. The body 50 may include an internal bore 70 for accommodating the suture 16. However, other configurations are contemplated within the scope of this disclosure.

The suture 16 may be passed through the second tissue 46 and then through the first tissue 44 after the surgical fixation device 48 has been positioned/implanted relative to the second tissue 46. The suture 16 may then be accommodated through the cannulation 28 of the locking ferrule 14, which as shown, may be pre-positioned within the opening 22 of the surgical fixation device 12.

Thereafter, the suture 16 may be tensioned in the first direction D1 to shorten a length L of the suture 16 extending between the surgical fixation devices 12, 48. The suture 16 may be tensioned to achieve a desired amount of compression between the first and second tissues 44, 46, thereby drawing the tissues toward one another or even into direct contact with one another if desired. The locking barbs 34 of the locking ferrule 14 prevent the suture 16 from sliding back in the second direction D2 subsequent to tensioning in the first direction D1. Furthermore, the cap portion 24 prevents the locking ferrule 14 from pulling through the surgical fixation device 12 during and subsequent to the tensioning.

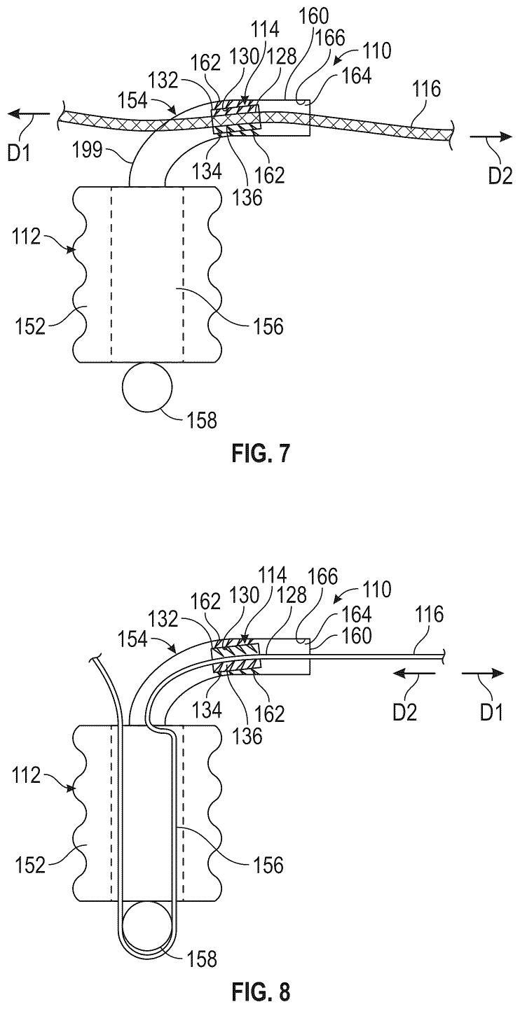

FIGS. 7 and 8 illustrate another exemplary suture locking system 110 that can be utilized to tension and lock a suture 116 during a surgical procedure, such as a tissue repair, for example. The suture locking system 110 may include a suture anchor 112, a locking ferrule 114, and one or more strands of the suture 116.

The suture anchor 112 may include a body portion 152 and a suture portion 154 that is connected to the body portion 152. The body portion 152 of the suture anchor 112 may be made from any biocompatible material, including but not limited to suitable polymeric materials or metallic materials. The body portion 152 may include a cannulated passage 156. In an embodiment, the cannulated passage 156 extends entirely through the body portion 152.

The suture portion 154 may be accommodated within the cannulated passage 156. A knot 158 of the suture portion 154 may retain the suture portion 154 within the cannulated passage 156 and thus relative to the body portion 152. A tail 160 of the suture portion 154 may extend outside of the body portion 152. In an embodiment, the knot 158 and the tail 160 of the suture portion 154 are located at opposite ends of the body portion 152. However, other configurations are contemplated within the scope of this disclosure.

The locking ferrule 114 may include an inner diameter wall 130 and an outer diameter wall 132. A cannulation 128 may extend through the locking ferrule 114 and may establish an internal passageway for accommodating one or more strands of the suture 116. The cannulation 128 may be circumscribed by the inner diameter wall 130.

A plurality of locking barbs 134 may protrude inwardly from the inner diameter wall 130. The locking barbs 134 may therefore occupy at least a portion of the open space of the cannulation 128. Each locking barb 134 may include a sharp or pointed tip 136, and, in this implementation, each locking barb 134 may angled in a common direction within the cannulation 128. The locking barbs 134 may therefore establish a one-way locking mechanism that permits the suture 116 to pass through the cannulation 128 in a first direction D1 while preventing the suture 116 from being tensioned or otherwise moved in a second direction D2.

The locking ferrule 114 may be inserted into a lumen 164 of the tail 160 of the suture portion 154 of the suture anchor 112. Another plurality of barbs 162 may protrude outwardly from the outer diameter wall 132 of the locking ferrule 114. The plurality of barbs 162 are configured to engage an internal wall 166 of the tail 160 that circumscribes the lumen 164 for locking a position of the locking ferrule 114 within the lumen 164.

In the embodiment of FIG. 7, the suture 116 may be shuttled into the lumen 164 and then through the cannulation 128 of the locking ferrule 114 before exiting back through an outer jacket 199 of the tail 160 of the suture portion 154. The suture 116 may then be tensioned in the direction D1 to allow the pointed tips 136 of the locking barbs 134 to interdigitate with the section of the suture 116 accommodated within the cannulation 128, thereby locking the suture 116 in place and preventing it from sliding back toward the second direction D2.

In the embodiment of FIG. 8, the suture 116 may be shuttled through the cannulated passage 156 of the body portion 152 prior to passing the suture 116 into the lumen 164 of the tail 160 and then through the cannulation 128 of the locking ferrule 114. The suture 116 may then be tensioned in the first direction D1 to allow the pointed tips 136 of the locking barbs 134 to interdigitate with the section of the suture 116 accommodated within the cannulation 128, thereby locking the suture 116 in place and preventing it from sliding back toward the second direction D2.

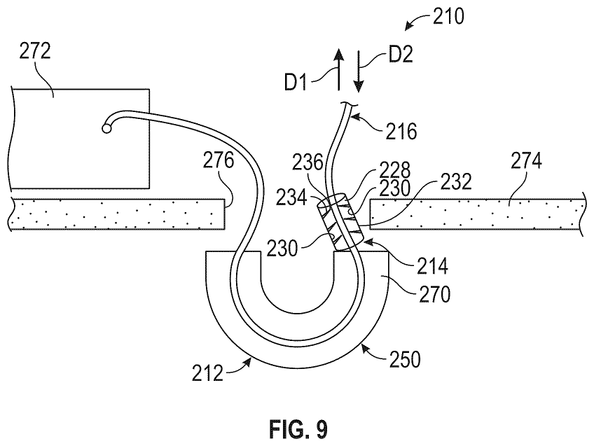

FIG. 9 illustrates another exemplary suture locking system 210 that can be utilized to tension and lock a suture 216 during a surgical repair (e.g., soft tissue-to-bone fixation, etc.). The suture locking system 210 may include a suture anchor 212, a locking ferrule 214, and the suture 216.

The suture anchor 212 may include a body 250. The body 250 may be a sheath made exclusively of soft, suture-based materials. However, other configurations are contemplated within the scope of this disclosure. The suture 216 may be passed through an internal bore 270 of the body 250 to connect the suture 216 to the suture anchor 212.

The locking ferrule 214 may include an inner diameter wall 230 and an outer diameter wall 232. A cannulation 228 may extend through the locking ferrule 214 and may establish an internal passageway for accommodating one or more strands of the suture 216. The cannulation 228 may be circumscribed by the inner diameter wall 230.

A plurality of locking barbs 234 may protrude inwardly from the inner diameter wall 230. The locking barbs 234 may therefore occupy at least a portion of the open space of the cannulation 228. Each locking barb 234 may include a sharp or pointed tip 236, and, in this implementation, each locking barb 234 may be angled in a common direction within the cannulation 228. The locking barbs 234 may therefore establish a one-way locking mechanism that permits the suture 216 to pass through the cannulation 228 in a first direction D1 while preventing the suture 216 from being tensioned or otherwise moved in a second direction D2.

The suture locking system 210 described above can be used to perform a variety of surgical methods, such as a method for attaching a tissue 272 (e.g., ligament, tendon, muscle, etc.) to a bone 274, for example. The tissue 272 may have torn away from the bone 274 during vigorous exercise or sporting activities, for example. When such tears occur, reattachment is often necessary to repair the tissue defect.

An exemplary surgical method may include preparing a socket 276 in the bone 274. The socket 276 may be a preformed opening formed in the bone 274 that is sized for receiving the suture anchor 212.

The suture 216 may be passed through or around the tissue 272 and may then be shuttled through both the internal bore 270 of the body 250 and the cannulation 228 of the locking ferrule 214. The suture 216 may then be tensioned in the direction D1 to knotlessly secure the tissue 272 at a desired position relative to the bone 274. Tensioning the suture 216 in the first direction D1 allows the pointed tips 236 of the locking barbs 234 to interdigitate with the portion of the suture 216 accommodated within the cannulation 228, thereby locking the suture 216 in place and preventing it from sliding back in the second direction D2. The locking ferrule 214 may be positioned within the socket 276 at a position that overlies the suture anchor 212 either during or subsequent to tensioning the suture 216 in the first direction D1.

The suture locking systems of this disclosure may be utilized for performing various tensionable knotless surgical repairs. The suture locking systems provide for tensioning and retensioning suture(s) at various points of the repair, including subsequent to implantation of accompanying fixation devices, thus providing numerous advantages over prior tissue repair systems and techniques.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A suture locking system, comprising:
a surgical fixation device including an opening;
a locking ferrule insertable within the opening and including a one-way locking mechanism provided within a cannulation of the locking ferrule; and
a suture received through the cannulation,
wherein the one-way locking mechanism is configured to allow the suture to be tensioned in a first direction while preventing the suture from moving in a second direction.

2. The suture locking system as recited in claim 1, wherein the surgical fixation device is a surgical button.

3. The suture locking system as recited in claim 1, wherein the surgical fixation device is a bone plate.

4. The suture locking system as recited in claim 1, wherein the suture is connected to a second surgical fixation device.

5. The suture locking system as recited in claim 4, wherein the second surgical fixation device is a suture anchor.

6. The suture locking system as recited in claim 1, wherein the locking ferrule includes a cap portion and a barrel portion, and further wherein the barrel portion is received through the opening and the cap portion is oversized relative to the opening.

7. The suture locking system as recited in claim 1, wherein the one-way locking mechanism is established by a plurality of locking barbs provided within the cannulation.

8. A suture locking system, comprising:
a suture anchor including a body portion and a suture portion connected to the body portion;
a locking ferrule received within a lumen of a tail of the suture portion and including a one-way locking mechanism provided within a cannulation of the locking ferrule; and
a suture received through the cannulation,
wherein the one-way locking mechanism is configured to allow the suture to be tensioned in a first direction and prevent the suture from moving in a second direction.

9. The suture locking system as recited in claim 8, wherein a plurality of locking barbs protrude inwardly from an inner diameter wall of the locking ferrule, and further comprising a plurality of barbs that protrude outwardly from an outer diameter wall of the locking ferrule to engage an interior wall of the tail.

10. The suture locking system as recited in claim 8, wherein the suture extends through a cannulated passage of the body portion of the suture anchor.

11. The suture locking system as recited in claim 8, wherein a knot of the suture portion secures the suture portion to the body portion of the suture anchor.

12. The suture locking system as recited in claim 11, wherein the knot and the tail are located at opposite ends of the body portion.

13. A suture locking system, comprising:
a suture anchor including a sheath having an internal bore;
a locking ferrule including a one-way locking mechanism; and
a suture received through the internal bore of the sheath and the locking ferrule.

14. The suture locking system as recited in claim 13, wherein the one-way locking mechanism allows the suture to be tensioned in a first direction and prevents the suture from moving in a second direction relative to the locking ferrule.

15. The suture locking system as recited in claim 13, wherein the one-way locking mechanism is established by a plurality of locking barbs provided within a cannulation of the locking ferrule.

16. The suture locking system as recited in claim 13, wherein the sheath is comprised exclusively of soft, suture-based materials.
